(19) United States
(12) Patent Application Publication
(10) Pub. No.: US 2026/0021292 A1
(43) Pub. Date: Jan. 22, 2026

(54) SYSTEM FOR PLACEMENT OF A MEDICAL DEVICE INCLUDING AN INFLATABLE BALLOON

(71) Applicant: Medtronic, Inc., Minneapolis, MN (US)

(72) Inventors: Kevin R. Seifert, Forest Lake, MN (US); Claire A. Winchester, Columbia Heights, MN (US)

(21) Appl. No.: 18/997,756

(22) PCT Filed: Jul. 27, 2023
(86) PCT No.: PCT/US2023/028774

§ 371 Date: Jan. 22, 2025

Related U.S. Application Data
(60) Provisional application No. 63/369,628, filed on Jul. 27, 2022.

(51) Int. Cl. A61N 1/05; A61B 17/00; A61B 17/34; A61B 90/00

(52) U.S. Cl. CPC A61N 1/0573; A61B 17/3468; A61B 2017/00557; A61B 2090/0811; A61B 2090/3966; A61N 2001/058

(57) ABSTRACT

An implant tool system includes an implantable medical device. The implantable medical device includes a zone visual marker positioned on an exterior surface of the device. The zone visual marker includes a distal end of the zone visual marker. The distal end of the zone visual marker aligning with the proximal end of an introducer indicates when to withdraw the introducer. The zone visual marker further includes a proximal end of the zone visual marker. The proximal end of the zone visual marker aligning with the proximal end of the introducer indicates when to inflate a balloon carried by the device and affix the distal end of the implantable medical device to the target site via a fixation mechanism.

This application claims the benefit of U.S. Provisional Application Ser. No. 63/369,628, filed Jul. 27, 2022, the entire content of which is incorporated herein by reference.

### TECHNICAL FIELD

This disclosure relates generally to tool systems for implanting medical devices.

### BACKGROUND

Some types of implantable medical devices (IMDs), such as cardiac pacemakers or implantable cardioverter defibrillators systems, may be used to provide sensing and therapy for a patient via one or more electrodes. Some IMDs include one or more implantable medical electrical leads that include one or more electrodes. The leads may be configured such that the electrodes may be implanted at target locations for sensing and/or therapy, such as within the heart (e.g., transvenous leads) or outside of the heart and vasculature (e.g., extravascular leads) for cardiac sensing and/or therapy. Once the leads are implanted, tines or other fixation elements attached to various locations of the leads may be deployed to prevent the leads from shifting or moving.

### SUMMARY

In some examples, an implant tool system comprises: an implantable medical device sized to be inserted through a lumen of an introducer from a proximal end of the introducer to a distal end of the introducer, wherein the implantable medical device comprises: a device body extending from a proximal end of the implantable medical device to a distal end of the implantable medical device; a balloon mechanically supported by the device body; a fixation mechanism configured to affix the distal end of the implantable medical device to a target site within a body of a patient; and a zone visual marker, positioned on an exterior surface of the device body, comprising: a distal end of the zone visual marker, wherein the distal end of the zone visual marker aligning with the proximal end of the introducer indicates when to withdraw the introducer; and a proximal end of the zone visual marker, wherein the proximal end of the zone visual marker aligning with the proximal end of the introducer indicates when to inflate the balloon and affix the distal end of the implantable medical device to the target site via the fixation mechanism.

In some examples, a method comprises: inserting an introducer through an access point on a body of a patient, wherein the introducer defines a lumen extending from a proximal end of the introducer to a distal end of the introducer; inserting an implantable medical device through the lumen until a distal end of a zone visual marker, positioned on an exterior surface of a device body of the implantable medical device, aligns with the proximal end of the introducer; withdrawing the introducer at least until a proximal end of the zone visual marker aligns with the proximal end of the introducer; inflating a balloon mechanically supported by device body; and affixing, via a fixation mechanism, the distal end of the implantable medical device to a target site within the body of the patient.

The details of one or more examples are set forth in the accompanying drawings and the description below. Other features, objects, and advantages will be apparent from the description and drawings, and from the claims.

### BRIEF DESCRIPTION OF THE DRAWINGS

FIG. 1A is a conceptual drawing illustrating an example implant tool system including a zone visual marker in accordance with techniques of this disclosure.

FIG. 1B is a conceptual drawing illustrating an example implantable medical balloon lead and an example introducer in accordance with techniques of this disclosure.

FIG. 2 is a conceptual drawing illustrating an example implantable medical balloon lead and an example introducer in accordance with techniques of this disclosure.

FIG. 3 is a conceptual drawing illustrating an example implantable medical lead in accordance with techniques of this disclosure.

FIG. 4 is a flow diagram of an example technique for delivering an appropriate length of lead into a patient's body.

FIG. 5 is a flow diagram of an example technique for delivering an appropriate length of lead into a patient's body.

### DETAILED DESCRIPTION

During implantation of an implantable lead including an inflatable balloon, a clinician may use a sheath introducer (“introducer”) to gain access of the IMD into vasculature of a patient. As part of the procedure, the clinician may advance a distal end of the lead, which supports the balloon, beyond a distal end of the introducer so that the balloon can be properly inflated (e.g., without damaging the balloon). To visualize the lead, the clinician may use fluoroscopic imaging to determine when the balloon has been advanced beyond the distal end of the introducer.

In the event the distal end of the lead needs to be withdrawn into the introducer (e.g., to check the balloon), the balloon may need to be deflated prior to being pulled into the introducer; otherwise, the balloon can be damaged. Determining where the inflated balloon is relative to the introducer can be difficult and often requires additional use of fluoroscopic imaging, which may be detrimental to the health of a patient.

In accordance with techniques of this disclosure, a lead may include one or more visual markers that indicate the position of the lead relative to an introducer without the use of visualization techniques that may adversely affect a patient, such as fluoroscopy. The lead may support the visual markers. When outside the patient's body, the visual markers may be visible to the naked eye; when inside the patient's body, the visual markers may be visible in ultrasound and other imaging techniques with fewer risks to a patient than fluoroscopy. In this way, the techniques of this disclosure may provide information that helps a clinician to more safely and consistently implant a lead.

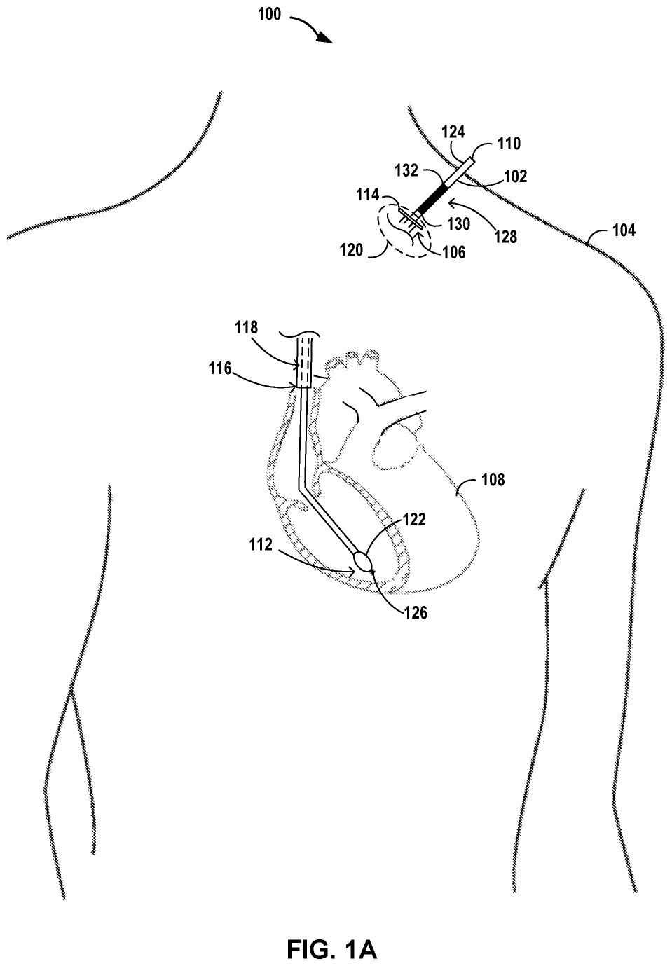

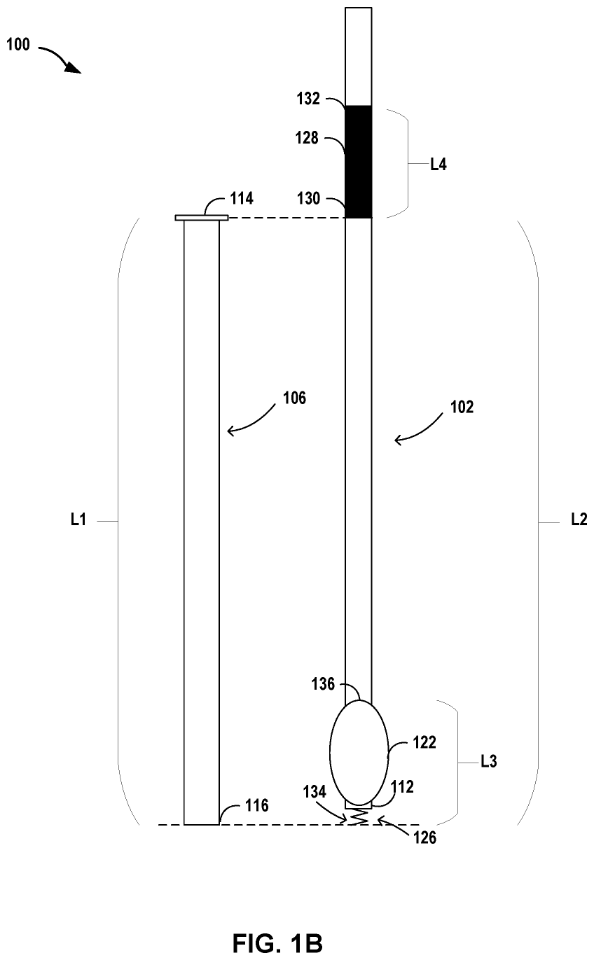

FIGS. 1A-1B are conceptual diagrams of an example implant tool system 100 (“system 100”) for implanting an implantable medical device (IMD), such as implantable medical balloon lead 102 (“lead 102”), within a patient 104. As shown in FIGS. 1A-1B, system 100 includes lead 102 that provides pacing therapy and a sheath introducer 106 (“introducer 106”).

Some of the components of system 100 described herein, such as one or more visual markers, may be utilized with other types of implant tool systems, such as implant tool systems for delivering IMDs configured to deliver electrical therapy other than cardiac electric therapy. For example, the techniques of this disclosure may be applied to non-cardiac contexts, such as neurostimulation. Furthermore, although primarily described herein as being an implantable medical lead (e.g., a cardiac lead), the IMD may be another type of device, such as a catheter. In addition, it should be noted that system 100 may not be limited to treatment of a human patient. System 100 may be implemented in non-human patients, such as primates, canines, equines, pigs, ovines, bovines, felines, etc. These non-human patients may undergo clinical or research therapies that may benefit from the subject matter of this disclosure.

In general, a clinician may insert lead 102 into and through a patient's vasculature to a target site within a body of patient 104 (e.g., tissue of a heart 108 of patient 104) where a medical procedure may be undertaken. Lead 102 may have a proximal end 110 (“lead proximal end 110”) and a distal end 112 (“lead distal end 112”). Lead 102 may be temporary (e.g., lead 102 may be a temporary pacing balloon lead) or permanent. The length of lead 102 may vary. In some examples, lead 102 may be about 100 centimeters (cm).

Introducer 106 may have a proximal end 114 (“introducer proximal end 114”) and a distal end 116 (“introducer distal end 116”). Introducer 106 may define a lumen 118 extending from introducer proximal end 114 to introducer distal end 116. Lumen 118 may be sized to allow lead 102 to be inserted through lumen 118 from introducer proximal end 114 to introducer distal end 116. The length of introducer 106 may vary. In some examples, the length of introducer 106 may be between about 10 cm and 25 cm. Introducer proximal end 114 may be exposed at an access point 120. That is, a portion of introducer 106 may be intracorporeal, while a remaining portion of introducer 106 may be extracorporeal.

Lead 102 may include a balloon 122 mechanically supported by a body 124 of lead 102 (“lead body 124”). Lead body 124 may extend from lead proximal end 110 to lead distal end 112. Balloon 122 may be located proximate (e.g., near) lead distal end 112. Balloon 122 may define an interior volume configured to receive an inflating medium (e.g., air, saline, or another medium), in turn resulting in inflation of balloon 122. A pump, such as a syringe, in fluid communication with balloon 122 may deliver the inflating medium to balloon 122. During an implantation procedure, a clinician may advance balloon 122 such that balloon 122 is positioned completely outside of introducer 106 to prevent contact between balloon 122 when in an inflated configuration and introducer 106. Once balloon 122 is positioned outside of introducer 106, the clinician may inflate balloon 122.

In examples, lead body 124 defines an inflation lumen fluidly coupled to the interior volume and configured such that a clinician may deliver the inflating medium to the interior volume defined by balloon 122. The inflation lumen may extend from a distal portion to a proximal portion of lead body 124. An exterior surface of lead body 124 may define an opening to the inflation lumen. For example, lead proximal end 110 may define an opening to the inflation lumen, such that the inflation lumen extends from balloon 122 to the opening.

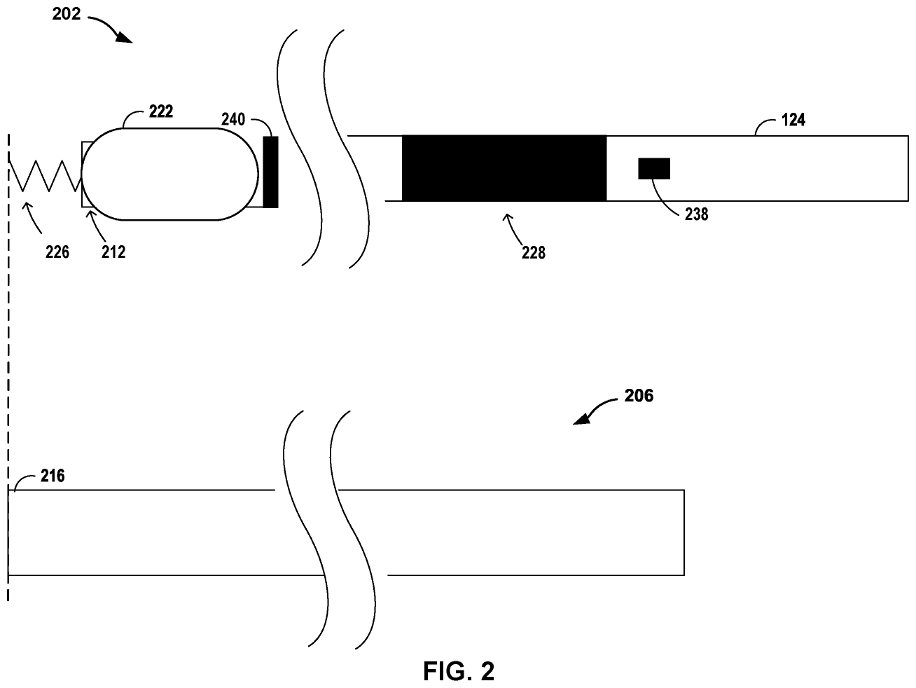

Lead 102 may include a fixation mechanism 126 to prevent lead 102 from shifting or moving (e.g., in a proximal direction) once fixed to tissue at a target location. Fixation mechanism 126 may be configured to engage tissue of heart 108. Fixation mechanism 126 may include, for example, one or more elongated tines such as fixation tines configured to substantially maintain an orientation of lead 102 with respect to a target site. Fixation mechanism 126 may include any shape, including a helically-shaped fixation element, as shown in FIGS. 1-2. Fixation mechanism 126 may be attached to lead distal end 112. Fixation mechanism may be fixed at lead distal end 112, or may be retractable/advanceable relative to lead distal end 112.

In accordance with techniques of this disclosure, lead 102 may include one or more visual markers that a clinician may visually reference and potentially feel during an implantation procedure to help the clinician determine when to inflate balloon 122. For example, system 100 may include at least one zone visual marker 128 positioned on the exterior surface of lead body 124. In some examples, zone visual marker 128 may be integrated into lead body 124. For example, zone visual marker 128 may be printed, sprayed, written, or otherwise impressed upon the exterior surface of lead body 124. Zone visual marker 128 may not materially change the overall diameter of lead 102, although zone visual marker 128 may optionally have a substantial width, depth, and/or density sufficient to make zone visual marker 128 visible. Furthermore, although shown as a band in FIG. 1A, zone visual marker 128 may possess any shape. Thus, other shapes of zone visual marker 128, such as a marker in the shape of a circle (e.g., a dot), a line (e.g., a dash), a cross (e.g., crosshatching), and/or the like, are contemplated. Additionally, zone visual marker 128 may be labeled, e.g., with words, letters, pictures, or symbols, colored, etc.

Zone visual marker 128 may be positioned on lead body 124 in accordance with techniques of this disclosure to help a clinician determine when to inflate balloon 122. For example, zone visual marker 128 may be positioned such that alignment of a distal end 130 of zone visual marker 128 (“zone distal end 130”) with introducer proximal end 114 indicates that lead distal end 112 and introducer distal end 116 are aligned. Accordingly, alignment of zone distal end 130 with introducer proximal end 114 may indicate when to withdraw introducer 106 from patient 104 (e.g., to allow balloon 122 to inflate without contacting introducer 106) or otherwise move introducer 106 relative to lead 102.

In addition, zone visual marker 128 may be positioned such that alignment of a proximal end 132 of zone visual marker 128 (“zone proximal end 132”) with introducer proximal end 114 may indicate when balloon 122 is positioned completely outside of introducer 106. In some examples, the longitudinal length of zone visual marker 128 (i.e., the distance from zone proximal end 132 to zone distal end 130) may substantially correspond to (e.g., be about equal to) the longitudinal length from a proximal end of balloon 122 to lead distal end 112. Accordingly, alignment of zone proximal end 132 with introducer proximal end 114 may indicate when to inflate balloon 122. It should be understood that introducer 106 may be withdrawn even more such that zone proximal end 132 is distal to introducer proximal end 114. Zone proximal end 132 being distal to introducer proximal end 114 may also indicate when to inflate balloon 122. In some examples, alignment of zone proximal end 132 with introducer proximal end 114 may indicate when to deflate balloon 122 such that balloon 122 can be withdrawn through introducer 106.

As shown in FIG. 1A, zone visual marker 128 is extracorporeal. Thus, a clinician may see zone visual marker 128 with the naked eye (e.g., without the use of imaging techniques, such as fluoroscopy). In this way, the techniques of this disclosure allow a clinician to implant lead 102 without the usage of imaging techniques that may adversely affect a patient, increasing patient safety while potentially improving the rate of successful implantation procedures.

Although illustrated in FIG. 1A as only supporting one zone visual marker 128, lead 102 may support a plurality of zone visual markers 128. This configuration may be advantageous because the type of introducer 106 required for a medical procedure may depend on the medical procedure. As discussed above, different introducers 106 may vary in length. Consequently, lead 102 may support a plurality of zone visual markers 128, where each zone visual marker 128 is compatible with a type of introducer 106 having a certain length. Each zone visual marker 128 may have a unique label, color, or other visual marking to facilitate identification of the appropriate zone visual marker for a type of introducer 106.

FIG. 1B is a conceptual drawing illustrating lead 102 and introducer 106. Introducer 106 may be configured to guide another component of system 100, such as a guidewire (e.g., a thin, flexible, medical wire), along an implantation path within a patient's body. For example, a guidewire may be inserted through lumen 118 and advanced to a target site. Introducer 106 may then translate along the guidewire.

A longitudinal length LI of introducer 106 may substantially correspond to a longitudinal length of lead 102 extending from zone distal end 130 to a distal end 134 of fixation mechanism 126 (“fixation mechanism distal end 134”). Accordingly, when introducer distal end 116 and fixation mechanism distal end 134 are aligned, introducer proximal end 114 and zone distal end 130 may also be aligned. As a result, fixation mechanism 126 may be protected within introducer 106 until the clinician begins to withdraw introducer 106. A longitudinal length L3 extending from a proximal end 136 of balloon 122 (“balloon proximal end 136”) to fixation mechanism distal end 134 may substantially correspond to a longitudinal length L4 of zone visual marker 128 (i.e., the distance from zone proximal end 132 to zone distal end 130). Accordingly, when zone proximal end 132 and introducer proximal end 114 are aligned, balloon 122 may be positioned completely outside of introducer 106 such that balloon 122 may inflate without contacting introducer 106.

FIG. 2 is a conceptual diagram of an example implantable medical balloon lead 202 (“lead 202”) that includes a zone visual marker 228 and an example sheath introducer 206 (“introducer 206”). Lead 202 may be substantially similar to lead 102 shown in FIG. 1. Introducer 206 may be substantially similar to introducer 106 shown in FIG. 1. As shown in FIG. 2, a body 224 of lead 202 (“lead body 224”) mechanically supports a balloon 222 proximate a distal end 212 of lead 202 (“lead distal end 212”). For example, a distal portion of lead 202 may mechanically support balloon 222. As further shown in FIG. 2, a fixation mechanism 226 may be attached to lead distal end 212.

Lead 202 may include one or more visual markers in addition to zone visual marker 228. For example, lead 202 may include at least one rotation visual marker 238. Rotation visual marker 238 may be positioned on the exterior surface of lead body 224. In some examples, rotation visual marker 238 may be a longitudinal visual marker. For instance, rotation visual marker 238 may extend around only a portion of the circumference of lead body 224. In this way, a clinician may visually determine angular rotation of rotation visual marker 238 (e.g., by counting revolutions of rotation visual marker 238). An angular rotation of rotation visual marker 238 may correspond to an angular rotation of fixation mechanism 226. For example, one revolution of rotation visual marker 238 may correspond to one revolution of fixation mechanism 226 (e.g., because lead 202 is rigid and flexes a negligible amount under torsion). In another example, one revolution of rotation visual marker 238 may correspond to less than one revolution of fixation mechanism 226 (e.g., because lead 202 is not rigid and flexes a significant amount under torsion). In any case, a relationship between the angular rotation of rotation visual marker 238 and the angular rotation of fixation mechanism 226 may be determined.

A clinician may use the angular rotation of rotation visual marker 238 to determine the degree to which fixation mechanism 226 has been rotated. This may be advantageous when a clinician rotates fixation mechanism 226 (which may be a helix or otherwise helically-shaped) to secure fixation mechanism 226 to tissue. For example, fixation mechanism 226 may need to revolve three times to be properly secured to tissue. If one revolution of rotation visual marker 238 corresponds to one revolution of fixation mechanism 226, a clinician may rotate lead 202 until the clinician observes rotation visual marker 238 complete three revolutions (in turn indicating that fixation mechanism 226 likewise revolved three times).

In some examples, lead 202 may include at least one radiopaque marker 240. Radiopaque marker 240 may be formed from radiopaque materials embedded within lead 202, respectively. In some examples, radiopaque marker 240 may be a band of radiopaque materials that runs along an outer circumference of a portion of lead 202. In some examples, fixation tine may be formed from or coated with radiopaque materials.

In any case, radiopaque marker 240 may indicate when to withdraw introducer 206 from a patient (e.g., to allow balloon 222 to inflate without contacting introducer 206). For example, radiopaque marker 240 and introducer 206 may be visible in fluoroscopy such that a clinician may visually determine a location of radiopaque marker 240 relative to a distal end 216 of introducer 206 (“introducer distal end 216”).

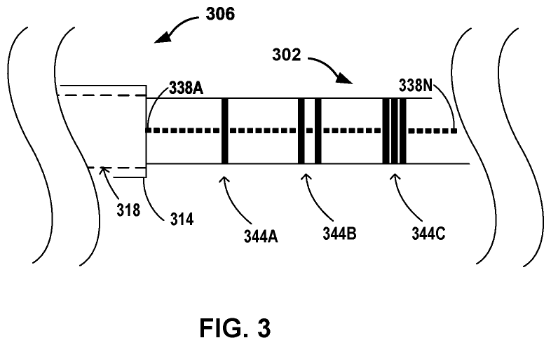

FIG. 3 is a conceptual diagram of an example implantable medical balloon lead 302 (“lead 302”). Lead 302 may be substantially similar to lead 102 shown in FIG. 1 and/or lead 202 shown in FIG. 2. As shown in FIG. 2, a body 324 of lead 302 (“lead body 324”) may include one or more visual markers, such as rotation visual markers 338A-338N (collectively, “rotation visual markers 338”) and depth visual markers 344A-344C (collectively, “depth visual markers 344”). In some examples, and as shown in FIG. 3, rotation visual markers 338 may indicate a series of measurement gradations. For example, rotation visual markers 338 may represent submarkings that are spaced apart at 1 cm increments. In this way, rotation visual markers 338 may help a clinician measure movement of lead 302. For instance, a clinician may introduce 3 cm of lead slack by advancing lead 302 three rotation visual markers 338 into introducer 306. For similar reasons, rotation visual markers 338 may help with management of lead 302. For example, rotation visual markers 338 may indicate when lead 302 is being pushed, pulled, rotated, etc., and a clinician may use this information to avoid dislodging 302.

Depth visual markers 344 may be positioned on an exterior surface of lead body 324. Depth visual markers 344 may be circumferential markers. For example, depth visual markers 344 may completely extend around the circumference of lead body 324. In some examples, rotation visual markers 338 may be positioned proximate depth visual markers 344. Depth visual markers 344 may indicate a depth of insertion of lead through a lumen 318 of an introducer 306. Introducer 306 may be substantially similar to introducer 106 shown in FIG. 1 and/or introducer 206 shown in FIG. 2.

During insertion of lead 302 into introducer 306, the entire length of lead 302 moves such that the visual markers of lead 302 likewise move. The clinician may determine the length of lead 302 inserted into introducer 306 by visual or manual reference to the relative movement of depth visual markers 344 to a reference point. In this way, depth visual markers 344 may help the clinician determine whether a sufficient amount of lead 302 has been introduced into patient's body.

For example, depth visual markers 344 may be positioned such that alignment of depth visual marker 344A with a proximal end 314 of introducer 306 (“introducer proximal end 314”) indicates a depth of insertion of lead 302 of a first length (e.g., 10 centimeters (cm)), alignment of depth visual marker 344B with introducer proximal end 314 indicates a depth of insertion of lead 302 of a second length (e.g., 20 cm), and alignment of depth visual marker 344C with introducer proximal end 314 indicates a depth of insertion of lead 302 of a third length (e.g., 30 cm). It should be understood that increments other than 10 cm (e.g., increments of 5 cm, 15 cm, etc.) are contemplated by this disclosure. Also, it should be understood that lead 302 may include more or fewer depth visual markers 344.

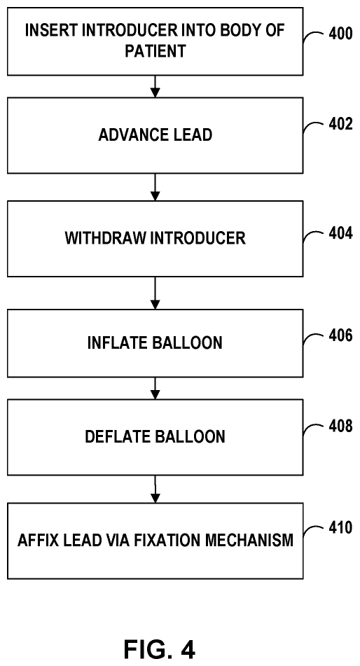

FIG. 4 is a flow diagram of an example technique for delivering an appropriate length of lead into a patient's body. Although FIG. 4 is discussed primarily in the context of system 100 of FIGS. 1A and 1B, it should be understood that the method of FIG. 4 may be applied to other examples of implant tool systems described herein.

A clinician (or other medical practitioner) may insert introducer 106 into patient's body (400). For example, the clinician may insert introducer 106 through access point 120. The clinician may advance introducer 106 along an implantation path. In some examples, advancement of introducer 106 (and/or other components of system 100) may be guided by ultrasound. By using ultrasound, a clinician may avoid or limit the usage of fluoroscopic observation, which may increase the safety of the implantation procedure.

The clinician may advance lead 102 into the patient's body (402). For example, the clinician may translate lead 102 relative to introducer 106 until zone distal end 130 and introducer proximal end 114 are aligned. In some examples, alignment of zone distal end 130 and introducer proximal end 114 may indicate that lead distal end 112 and introducer distal end 116 are aligned. In some examples, alignment of zone distal end 130 and introducer proximal end 114 may indicate that fixation mechanism distal end 134 and introducer distal end 116 are aligned. In any case, alignment of zone distal end 130 and introducer proximal end 114 may indicate when to withdraw introducer 106.

The clinician may withdraw introducer 106 (404). For example, the clinician may withdraw lead introducer 106 until zone proximal end 132 and introducer proximal end 114 are aligned. Alignment of zone proximal end 132 with introducer proximal end 114 may indicate that balloon 122 of lead 102 is positioned completely outside of introducer 106. Accordingly, alignment of zone proximal end 132 with introducer proximal end 114 may indicate that it is safe to inflate balloon 122.

Responsive to aligning zone proximal end 132 with introducer proximal end 114 (or withdrawing introducer 106 such that zone proximal end 132 is distal to introducer proximal end 114). It should be understood that introducer 106 may be withdrawn even more such that zone proximal end 132 is distal to introducer proximal end 114. Zone proximal end 132 being distal to introducer proximal end 114 may also indicate when to inflate balloon 122.

Responsive to zone proximal end 132 being aligned with or distal to introducer proximal end 114, the clinician may inflate balloon 122 without any possibility of balloon 122 contacting introducer 106 (406). In some examples, when balloon 122 is inflated, a clinician may electrically test one or more implant sites. Based on the signals detected from the implant sites, a clinician may select one as a target site for implantation. If lead distal end 112 is not already at or proximate the target site, the clinician may advance lead 102. Prior to affixing lead 102 to the target site, the clinician may deflate balloon 122 completely (408). The clinician may then affix lead 102 to the target site (410).

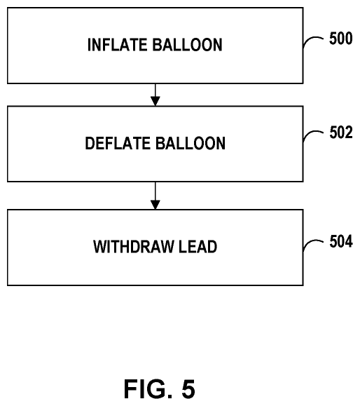

FIG. 5 is a flow diagram of an example technique for delivering an appropriate length of lead into a patient's body. Although FIG. 5 is discussed primarily in the context of system 100 of FIG. 1, it should be understood that the method of FIG. 5 may be applied to other examples of implant tool systems as described herein.

In some cases, a clinician may wish to inspect balloon 122 after it has been inflated. In such cases, it may be important to deflate balloon 122 when introducer 106 is not near balloon 122. Accordingly, zone proximal end 132 aligning with or being distal to introducer proximal end 114 may indicate when to deflate balloon 122 and withdraw implantable medical lead 102 through lumen 118.

Thus, after a clinician inflates balloon 122 (500), the clinician may check whether zone proximal end 132 is aligned with or distal to introducer proximal end 114. If zone proximal end 132 is aligned with or distal to introducer proximal end 114, the clinician may deflate balloon 122 (502). Once balloon 122 is fully deflated, the clinician may withdraw lead 102 through lumen 118 and from the patient's body (504).

The following examples are illustrative of the techniques described herein.
Example 1: An implant tool system includes an implantable medical device sized to be inserted through a lumen of an introducer from a proximal end of the introducer to a distal end of the introducer, wherein the implantable medical device includes: a device body extending from a proximal end of the implantable medical device to a distal end of the implantable medical device; a balloon mechanically supported by the device body; a fixation mechanism configured to affix the distal end of the implantable medical device to a target site within a body of a patient; and a zone visual marker, positioned on an exterior surface of the device body includes a distal end of the zone visual marker, wherein the distal end of the zone visual marker aligning with the proximal end of the introducer indicates when to move the introducer relative to the implantable medical device; and a proximal end of the zone visual marker, wherein the proximal end of the zone visual marker aligning with the proximal end of the introducer indicates when to: inflate the balloon and affix the distal end of the implantable medical device to the target site via the fixation mechanism, or deflate the balloon such that the balloon can be withdrawn through the introducer.
Example 2: The implant tool system of example 1, wherein the proximal end of the zone visual marker aligning with or being distal to the proximal end of the introducer indicates when to deflate the balloon and withdraw the implantable medical device through the lumen.
Example 3: The implant tool system of example 1 or 2, wherein the fixation mechanism is a helix.
Example 4: The implant tool system of any of examples 1 to 3, wherein the implantable medical device includes a rotation visual marker positioned on the exterior surface of the device body, wherein an angular rotation of the rotation visual marker corresponds to an angular rotation of the fixation mechanism.
Example 5: The implant tool system of example 4, wherein the rotation visual marker is a longitudinal visual marker that extends around only a portion of a circumference of the device body.
Example 6: The implant tool system of any of examples 1 to 5, wherein the implantable medical device includes one or more depth visual markers, positioned on the exterior surface of the device body, indicative of a depth of insertion of the implantable medical device through the lumen.
Example 7: The implant tool system of example 6, wherein the one or more depth visual markers are circumferential visual markers that completely extends around a circumference of the device body.
Example 8: The implant tool system of any of examples 1 to 7, wherein the implantable medical device includes at least one radiopaque marker that indicates when to move the introducer relative to the implantable medical device.
Example 9: The implant tool system of any of examples 1 to 8, wherein the zone visual marker is integrated into the device body.
Example 10: The implant tool system of any of examples 1 to 9, wherein the implantable medical device includes an implantable medical lead configured to deliver therapy to the patient.
Example 11: A method includes inserting an introducer through an access point on a body of a patient, wherein the introducer defines a lumen extending from a proximal end of the introducer to a distal end of the introducer; inserting an implantable medical device through the lumen until a distal end of a zone visual marker, positioned on an exterior surface of a device body of the implantable medical device, aligns with the proximal end of the introducer; moving the introducer relative to the implantable medical device at least until a proximal end of the zone visual marker aligns with the proximal end of the introducer; and inflating a balloon mechanically supported by device body, or deflating the balloon such that the balloon can be withdrawn through the introducer.
Example 12: The method of example 11, wherein the method further includes with the proximal end of the zone visual marker aligning with or being distal to the proximal end of the introducer, deflating the balloon; and withdrawing the implantable medical device through the lumen.
Example 13: The method of example 11 or 12, wherein the implantable medical device includes a fixation mechanism, and wherein the fixation mechanism is a helix.
Example 14: The method of any of examples 11 to 13, wherein the implantable medical device includes a rotation visual marker positioned on the exterior surface of the device body, wherein an angular rotation of the rotation visual marker corresponds to an angular rotation of the fixation mechanism, and wherein the method further includes: positioning the distal end of the implantable medical device such that the distal end of the implantable medical device abuts the target site; rotating the implantable medical device; and stopping rotation of the implantable medical device based on the angular rotation of the rotation visual marker.
Example 15: The method of example 14, wherein the rotation visual marker is a longitudinal visual marker that extends around only a portion of a circumference of a device body.
Example 16: The method of any of examples 11 to 15, wherein the implantable medical device includes one or more depth visual markers, positioned on the exterior surface of the device body, indicative of a depth of insertion of the implantable medical device through the lumen.
Example 17: The method of example 16, wherein the one or more depth visual markers are circumferential visual markers that completely extend around a portion of the device body.
Example 18: The method of any of examples 11 to 17, wherein the implantable medical device includes at least one radiopaque marker that indicates when to withdraw the introducer.
Example 19: The method of any of examples 11 to 18, wherein the zone visual marker is integrated into the device body.
Example 20: The method of any of examples 11 to 19, wherein the implantable medical device includes an implantable medical lead configured to deliver therapy to the patient.

Various aspects of the disclosure have been described. These and other aspects are within the scope of the following claims.

1. An implant tool system comprising:
an implantable medical device sized to be inserted through a lumen of an introducer from a proximal end of the introducer to a distal end of the introducer, wherein the implantable medical device comprises:
a device body extending from a proximal end of the implantable medical device to a distal end of the implantable medical device;
a balloon mechanically supported by the device body;
a fixation mechanism configured to affix the distal end of the implantable medical device to a target site within a body of a patient; and
a zone visual marker, positioned on an exterior surface of the device body, comprising:
a distal end of the zone visual marker, wherein the distal end of the zone visual marker aligning with the proximal end of the introducer indicates when to move the introducer relative to the implantable medical device; and
a proximal end of the zone visual marker, wherein the proximal end of the zone visual marker aligning with the proximal end of the introducer indicates when to:
inflate the balloon and affix the distal end of the implantable medical device to the target site via the fixation mechanism, or
deflate the balloon such that the balloon can be withdrawn through the introducer.

2. The implant tool system of claim 1, wherein the proximal end of the zone visual marker aligning with or being distal to the proximal end of the introducer indicates when to deflate the balloon and withdraw the implantable medical device through the lumen.

3. The implant tool system of claim 1, wherein the fixation mechanism is a helix.

4. The implant tool system of claim 1, wherein the implantable medical device comprises a rotation visual marker positioned on the exterior surface of the device body, wherein an angular rotation of the rotation visual marker corresponds to an angular rotation of the fixation mechanism.

5. The implant tool system of claim 4, wherein the rotation visual marker is a longitudinal visual marker that extends around only a portion of a circumference of the device body.

6. The implant tool system of claim 1, wherein the implantable medical device comprises one or more depth visual markers, positioned on the exterior surface of the device body, indicative of a depth of insertion of the implantable medical device through the lumen.

7. The implant tool system of claim 6, wherein the one or more depth visual markers are circumferential visual markers that completely extends around a circumference of the device body.

8. The implant tool system of claim 1, wherein the implantable medical device comprises at least one radiopaque marker that indicates when to move the introducer relative to the implantable medical device.

9. The implant tool system of claim 1, wherein the zone visual marker is integrated into the device body.

10. The implant tool system of claim 1, wherein the implantable medical device comprises an implantable medical lead configured to deliver therapy to the patient.

11. A method comprising:
inserting an introducer through an access point on a body of a patient, wherein the introducer defines a lumen extending from a proximal end of the introducer to a distal end of the introducer;
inserting an implantable medical device through the lumen until a distal end of a zone visual marker, positioned on an exterior surface of a device body of the implantable medical device, aligns with the proximal end of the introducer;
withdrawing the introducer at least until a proximal end of the zone visual marker aligns with the proximal end of the introducer;
inflating a balloon mechanically supported by the device body; and
affixing, via a fixation mechanism, the distal end of the implantable medical device to a target site within the body of the patient.

12. The method of claim 11, further comprising:
with the proximal end of the zone visual marker aligning with or being distal to the proximal end of the introducer, deflating the balloon; and
withdrawing the implantable medical device through the lumen.

13. The method of claim 11, wherein the fixation mechanism is a helix.

14. The method of claim 11, wherein the implantable medical device comprises a rotation visual marker positioned on the exterior surface of the device body, wherein an angular rotation of the rotation visual marker corresponds to an angular rotation of the fixation mechanism, and wherein affixing the distal end of the implantable medical device to the target site comprises:
positioning the distal end of the implantable medical device such that the distal end of the implantable medical device abuts the target site;
rotating the implantable medical device; and
stopping rotation of the implantable medical device based on the angular rotation of the rotation visual marker.

15. The method of claim 14, wherein the rotation visual marker is a longitudinal visual marker that extends around only a portion of a circumference of a device body.

16. The method of claim 11, wherein the implantable medical device comprises one or more depth visual markers, positioned on the exterior surface of the device body, indicative of a depth of insertion of the implantable medical device through the lumen.

17. The method of claim 16, wherein the one or more depth visual markers are circumferential visual markers that completely extend around a portion of the device body.

18. The method of claim 11, wherein the implantable medical device comprises at least one radiopaque marker that indicates when to withdraw the introducer.

19. The method of claim 11, wherein the zone visual marker is integrated into the device body.

20. The method of claim 11, wherein the implantable medical device comprises an implantable medical lead configured to deliver therapy to the patient.
